(19) United States
(12) Patent Application Publication
(10) Pub. No.: US 2014/0365157 A1
(43) Pub. Date: Dec. 11, 2014

(54) cartridge for test strips and a method for including a calibration code onto the cartridge and for reading the code

(71) Applicants: Vasa-Pekka Torvinen, Siivikkala (FI); Jarmo Maentausta, Espoo (FI); Jukka Planman, Helsinki (FI)

(72) Inventors: Vasa-Pekka Torvinen, Siivikkala (FI); Jarmo Maentausta, Espoo (FI); Jukka Planman, Helsinki (FI)

(73) Assignee: MENDOR OY, Espoo (FI)

(21) Appl. No.: 14/119,888

(22) PCT Filed: May 25, 2011
(86) PCT No.: PCT/FI2011/050477

§ 371 Date: Jul. 24, 2014

(51) Int. Cl. G01D 18/00; G01N 33/487

(52) U.S. Cl. CPC G01D 18/008; G01N 33/48707
USPC 702/104

(57) ABSTRACT

A cartridge for storing a plurality of test strips suitable for analyzing body fluid, such as blood is designed such that the test strips can be brought out of the cartridge for performing a body fluid measurement. Such test strips need to be identified in order to calibrate the measurement to lot-to-lot variations of test strips. The identification is accomplished by means for data transfer from the cartridge (4, 5) and an electrical component (9) connected between the electrical connectors for identifying a calibration code for the lot of strips included in the cartridge.

### FIELD OF THE INVENTION

The invention relates to body fluid measurement. In particular, the invention relates to a cartridge for storing a plurality of test strips suitable for analyzing body fluid, such as blood. Such cartridge is designed such that the test strips can be brought out of the cartridge for performing a body fluid measurement. Such test strips need to be identified in order to calibrate the measurement to lot-to-lot variations of test strips.

### BACKGROUND OF THE INVENTION

Disposable test strips are frequently used in personal blood glucose monitors for measuring blood glucose in daily life. Test strips are typically sensitive to outside elements, in particular humidity, that may compromise measuring accuracy after long-time exposure. Therefore, during long periods of storage, the test strips must be protected from outside elements, air humidity, in particular.

Traditionally, test strips have been stored in closeable plastic vials containing, for example, 25 to 50 strips. Such vials are not easy to use as they are separate components from the meter itself, resulting in more operations and hassle needed for performing a single blood glucose measurement. An exemplary vial is disclosed in WO 03/082092.

U.S. Pat. No. 6,908,008 discloses a test device with means for storing and dispensing test strips. The test strips are stored stacked within the device and pushed out by means of a slider.

Test strips may also be stored in cartridges (also cassettes, magazines) which are filled with test strips and placed into the monitor device. Some cartridges contain means for sealing each strip individually in a separate locker of the cartridge in a foil. Such cartridges are, however, expensive and difficult to manufacture. In addition, their dimensions are relatively large because of the individual lockers, resulting in bulky integrated monitors. Thus, they are not well suitable for small blood glucose monitors designed for frequently repeated measurements in everyday life.

Examples of commercially available test strip cartridges include Bayer Ascensia Breeze and Roche Accu-Chek Compact Plus. In patent literature, stacked test trip cartridges or the like devices have been disclosed, for example, in, WO 03/042691, U.S. Pat. No. 6,908,008, EP 1314029, CA 2583563.

Another type of cartridges (in addition to individually sealed strips) are cartridges that seal all strips inside the cartridge. This cartridge type removes the need for individual sealing. Typically the strips are stored in stacks. WO 2006/044850 and WO 2006/002432 disclose further examples of cartridges.

The test strip is actually a sensitive electrochemical biosensor. At the present test strips have wide lot-to-lot variations, whereby most of biosensors have some kind of calibration code that is predetermined in factory to compensate the error. This code can be included in the cartridge or the test strip itself or otherwise included in the package or information material included with new cartridges. If the code is not read automatically, it needs user activity to input the values to the monitor. I such case user may make input errors (wrong values are entered to the device), which may cause inaccurate results. Thus, an automatic coding method is needed, and consecutively, several auto coding methods have been introduced.

Obviously, optical or even mechanical bar code can be used, but it requires a bar code reader that increases the costs of the analyzing apparatus and the code itself needs rather lot of space on the cartridge. A galvanic connection coding matrix wherein a bit pattern is recognized on basis of connections between connector points is also in use. Resistance coding with analog-digital conversion for individual strips is described in WO200750396 for separate cartridge module in EP1729128. Optical methods can also be used and EEPROM memory code plates or other memory devices provide large storing capacity of various information and possibility to read and write to the memory. However, even though memory devices are rather inexpensive and small, they do increase the costs of the cartridge and require connections and devices for reading and writing the information that is handled.

US 2008/034834 discloses an arrangement wherein several resistors are connected to two concentric electrodes. A digital code can be established by using varying connections between the resistors and electrodes.

REVERTER F AT AL: “Accuracy and resolution of direct resistive sensor-to-microcontroller interfaces”, SENSORS AND ACTUATORS A. vol. 121, 31 May 2005, pages 78-87 discloses a method for measuring resistors by microcontrollers.

Since for example in treatment of diabetes measurement of blood sugar (glucose) has to be done several times daily, even minor increase in means for treating the condition amount considerable sums annually both for patient and the health care system. Therefore an inexpensive and reliable coding method is needed that can be implemented with elements requiring minimum space.

### SUMMARY OF THE INVENTION

It is an aim of the invention to produce a novel cartridge for test strips which includes an inexpensive system for storing a calibration code.

Another aim of one embodiment of the invention is to produce a cartridge having minimum connections for identifying the cartridge.

The invention also involves a method for including a calibration code to the cartridge.

The invention also involves a method for reading the calibration code as well as an arrangement for implementing the method.

According to one embodiment of the invention, the calibration code is stored in a circuit made on a printed circuit board.

According to one advantageous embodiment the invention utilizes time constant measurement for identifying a resistance value that is proportional to the code identifying the cartridge.

More specifically, the invention is characterized by what is stated in the independent claims.

The invention provides essential benefits.

According to one embodiment of the invention, the resistance is measured with time constant. When measuring the resistance with time (using timer of a microcontroller) in comparison to using the analog-digital conversion, the microcontroller can be more simple and low-end type, which makes it cheap and simple. Simple proven components are also reliable. The component amount needed to store and indicate the calibration code is minimized to two resistors and one capacitor. The solution is cheaper compared, for example, to the optical method or memory code plate. This method reserves or requires only three I/O pins from microcontroller. This means that less I/O pins are needed comparing for example to a coding matrix identification method, which provides for a cheap and simplified apparatus. Further, the connector that contacts to auto coding plate on the cartridge needs only two pins. Less pins on the connector are needed comparing to resistance coding matrix, which makes the connection area and connections cheap and small. Resistor in the device and resistor in the cartridge are only components that affect to the total code detection error. Thereby component errors and temperature coefficients are easier to manage comparing to AD-conversion, optical components, etc, providing the invention good detection accuracy compared to earlier methods. The apparatus needed and the measuring method is quite robust. Error in components depends on the component selection and the error can be high without causing code detection error. Since typically only 16 different coding values are needed to identify the calibration code, the steps between the resistor values may be relatively high. This gives a good resolution where different values are clearly visible do not easily mix with each other.

Still further embodiments and advantages of the invention are described in the following detailed description with reference to the attached drawings.

### BRIEF DESCRIPTION OF THE DRAWINGS

FIG. 1 shows a schematic drawing of the principle of the invention.

FIG. 2 shows schematically the implementation of one embodiment of the invention to an analyzing device and a cartridge.

FIG. 3 shows schematically from above one embodiment of a code plate that can be used in connection of the invention.

FIG. 4 shows the code plate of FIG. 1 in a side view.

FIG. 5 shows a perspective view of a cartridge that can be used for implementation of one embodiment of the invention.

FIG. 6 shows an exploded perspective view of a cartridge according to FIG. 6.

FIG. 7 shows a perspective view of a cartridge according to FIGS. 5 and 6 equipped with a test strip.

FIG. 8 shows one embodiment of the invention unassembled.

FIG. 9 shows the embodiment of FIG. 8 assembled.

### DETAILED DESCRIPTION OF EMBODIMENTS

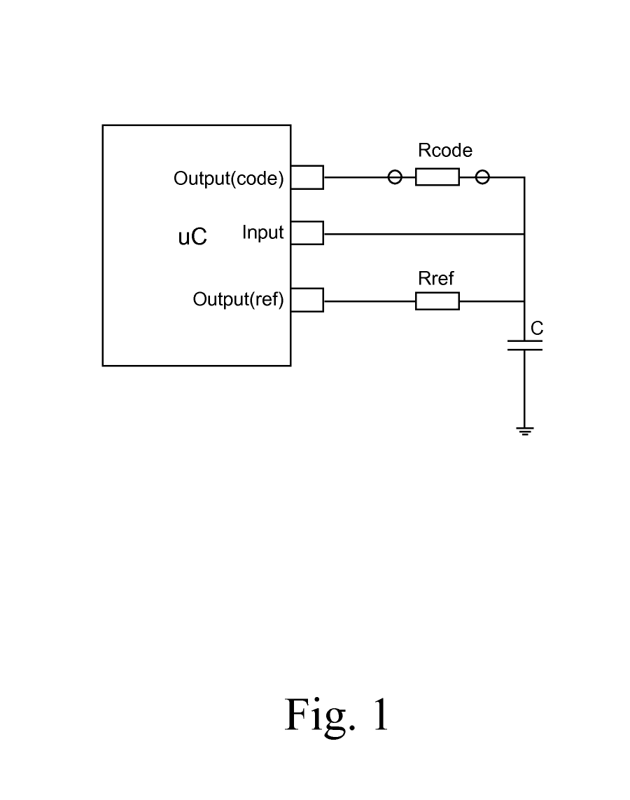

FIG. 1 shows one embodiment for circuitry for reading calibration code according to the invention. Herein a microcontroller uC is used for providing voltage for measurements an performing the calculation or detection of time. This embodiment uses one input pin (Input) for detecting incoming signal and two output pins (Output (code) and Output(ref) for providing voltage. On Output (code) pin it is connected a code resistor (Rcode) that is further connected to input pin and through a capacitator (C) to ground. On Output (ref) pin it is connected a reference resistor (Rref) that is also connected to same Input pin and through the same capacitator (C) to ground as the code resistor (Rcode). The resistance value of the code resistor is known.

Measurement and detection of the calibration code can be done with the above arrangement using following protocol:

1. Output(code) pin is set to high impedance state.

2. Output(ref) pin is set to ‘1’. At the same time the timer of time constant in microcontroller uC is reset.

3. When the input pin (Input) rises to ‘1’, the timer value of time constant is read and saved as the elapsed time of time(ref) variable.

4. Output(code) pin is set as output and set ‘0’.

5. Output(ref) pin is set to high impedance state.

6. Output(code) pin is set to ‘1’. At the same time the timer of time constant in microcontroller uC is reset.

7. When the input pin rises to ‘1’ the timer value of time constant is read and saved as elapsed time of time(code) variable.

8. Now, Rcode is calculated by following formula:

Rcode=time(code)/time(ref)

The resistance values of Rcodes are preferably selected so that they are multiplies of values of Rref. Then the calculation can be simplified to multiple decremention, which is more simplified calculation than division. A even more simple microcontroller can then be used.

9. Rcode value that is related autocode calibration value is read from matrix from any suitable memory device and this value is used in blood glucose calculations.

The theory of the measurement can also be explained by referring to FIG. 1.

The measurement method based on the time constant measurement via two resistors (Rcode and Rref) and one common capacitor (C). Both resistors are connected Output/high impedance pin in uC. When the output (Output[ref] or Output[code] has risen from 0V to 3V the input pin (Input) voltage will rise with delay that can be calculated with following formula:

V(input_trig)=V(output)*exp[−time/(R*C)]

In which,

V(input_trig) is a voltage level in a CMOS input when the input is seen as ‘1’ in uC

V(output) is voltage of output pin, which is in the beginning 0V and it will be rise with step to Vdd

Time is a time period from the rise of output 0V=3V to ‘1’ detection in input

R is a resistance of Rcode or Rref

C is a capacitance

Formulas for both time constant measurements are following:

V(input_trig)=V(output)*exp[−time(ref)/(Rref*C)]

V(input_trig)=V(output)*exp[−time(code)/(Rcode*C)]

Because the V(input_trig) is a voltage when digital output will detect voltage at ‘high’ stage and it is same for both measurement, which means that:

V(output)*exp[−time(ref)/(Rref*C)]=V(output)*exp[−time(code)/(Rcode*C)]

exp[−time(ref)/(Rref*C)]=exp[−time(code)/(Rcode*C)]

When taking ln from both side:

−time(ref)/(Rref*C)=−time(code)/(Rcode*C)

Rcode=Rref*time(code)/time(ref)

Rref can be selected as 1

Rcode=time(code)/time(ref).

The measurements can be made in opposite order.

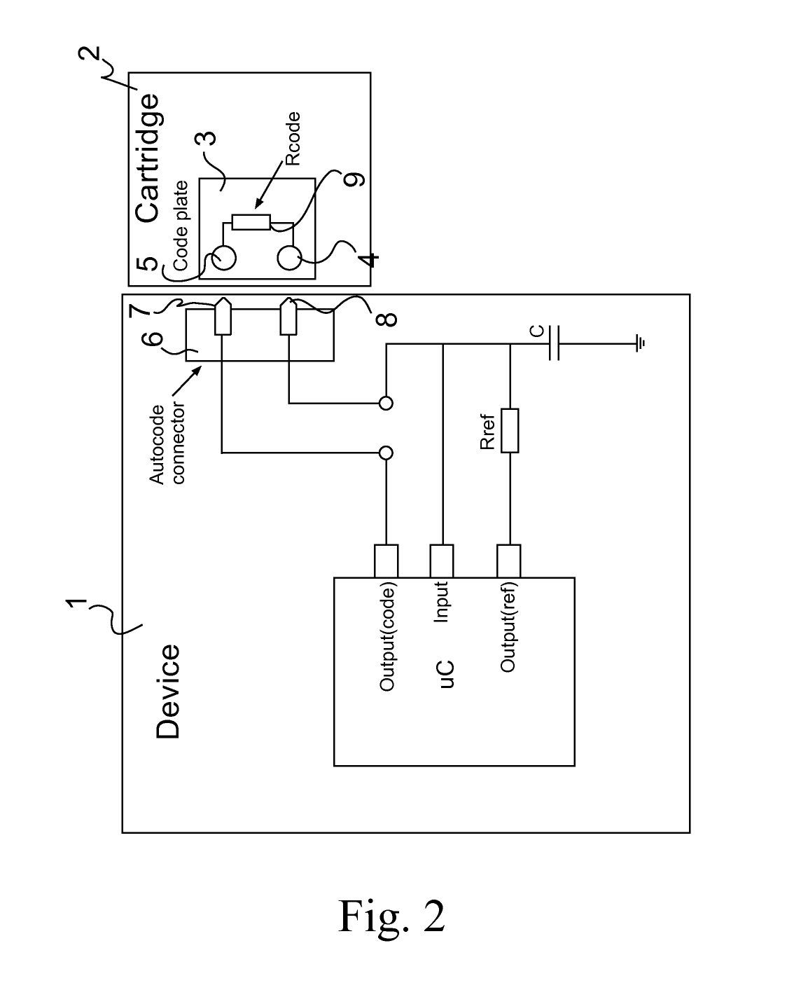

FIG. 2 shows one embodiment for implementation of the invention to a measuring device using a cartridge of test strips for doing the measurements. Herein the detection and measuring device is depicted simply as box 1 named “Device” and the cartridge is similarity depicted as a box 2 named “Cartridge”. Herein the cartridge includes a code plate 3 that includes first and second contact pin 4 5 and a resistor 9, that is the code resistor Rcode according to the invention. The resistor 9/Rcode is connected between the contact pins. The measuring apparatus 1 includes an autocode connector 6 that comprises connector pins 7, 8 corresponding those of the cartridges. When the cartridge is mounted on the measuring device 1, the connectors 4, 5, 7, 8, in autocode connector 6 and code plate 3 join the code resistor Rcode part of the code recognition circuitry of the measuring device as described above. When the connection is done, detection of the calibration code can be performed at any time before first test strip of the cartridge is used.

In this invention the autocoding method is based on electrical resistance. On the cartridge, an electronic component is installed, with a specific resistance value related to the characteristics of the test strips. In the embedded software or other processing means of the measuring apparatus, the resistance is measured, and the corresponding code value used to adapt the measurement to the characteristics of the lot of strips defined by the code.

Coding plate wherein the reference resistance Rref is arranged, is preferably a separate part of the cartridge, for example a PWB (printed wiring board) in which a resistor is assembled. Alternatively any other method for forming this simple circuitry may be used, as long as it is cost effective enough and forms a body that is separate from the cartridge but can be attached thereto. The PWB or other coding plate is mounted to the side of the test strip cartridge for example by gluing, heat or ultra sound welding, mechanical connectors or any other suitable method providing reliable attachment. The attachment should be preferably permanent in order to avoid loss of the code that would render the strips in the cartridge useless.

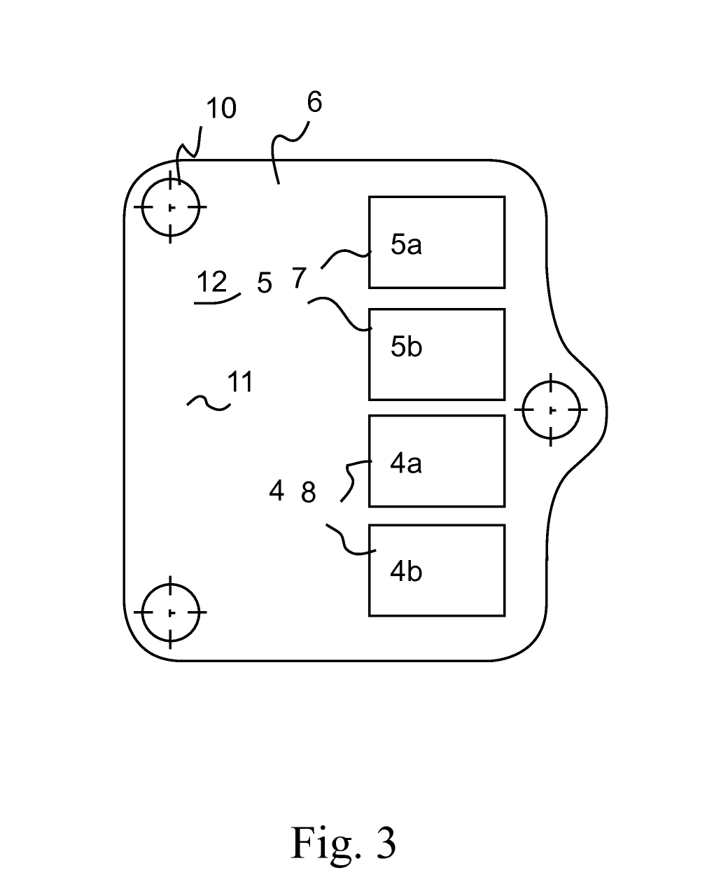

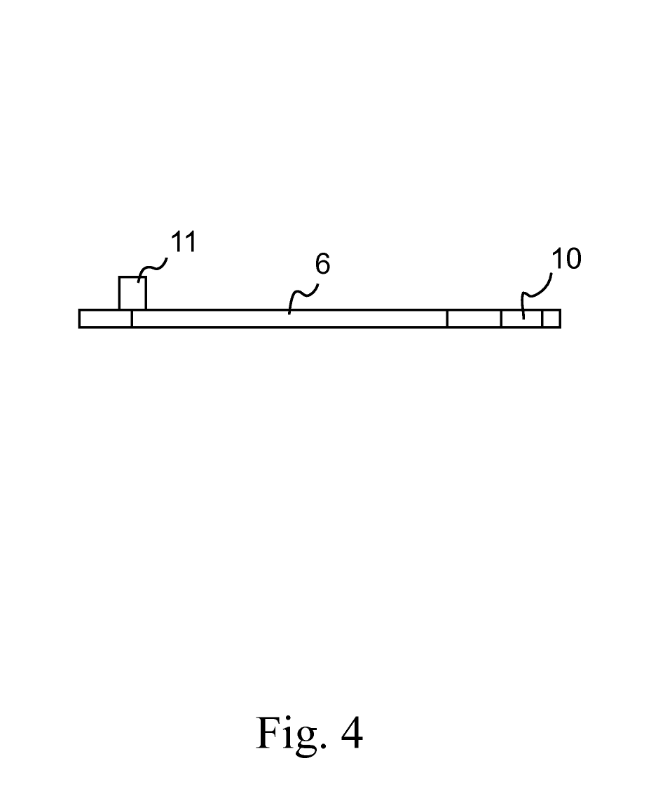

One embodiment of the code plate is shown in FIGS. 3 and 4. Herein the code plate 6 has a body part 12 of any suitably rigid material that has such electrical properties that the connectors and wiring can be made thereon. One possibility is above mentioned printed writing board. The body 12 is basically rectangular and has a curved extension on one side of the rectangle. Two of the corners and the extension of the body 12 have holes 10 for attaching the plate to a cartridge. On one long side of the rectangle is placed four contact surfaces. The surfaces are connected to pair wise to each other so that two contacts on each side form one connector 4, 5 having two contact surfaces 4a, 4b, 5a, 6b. This arrangement has the benefit that the connection to two separate surfaces is more reliable and if more connectors are needed, the wiring of the code plate can be redesigned to comprise four connectors. In this embodiment the attachment to the cartridge is accomplished by plastic pegs that penetrate the holes 10 and are pressed down by pressure and heat over the edges of the holes 10.

It can be contemplated that the resistance is mounted fixedly in the cartridge, but then either a method for setting the resistance value of the resistor is needed or one has to use several different cartridges having different resistors. This may cause confusion and undesired rise of costs.

In a preferred embodiment, the cartridge holds test strips in a stack format and is essentially formed as a space with a top part, four sides and a bottom part. In a preferred embodiment, the cartridge and meter have means to dispense a strip near the top of the cartridge, for example as described in PCT/FI/2010/050465, which is disclosed herein by reference. In a preferred embodiment the cartridge has means to facilitate an electrical contact between the test strips and device. The means can be an opening on the top of the device. In a preferred embodiment, the coding means are located on the top of the cartridge in the vicinity of the means for facilitating contact with the test strip. This placing of the elements makes it easier and more cost effective to establish the corresponding connectors in the measuring device. Furthermore, in a preferred embodiment, when the cartridge is placed in the measuring device, the cartridge is pressed by a force such as a spring, to keep both the test strip and coding connectors well connected to the device. In a preferred embodiment, the spring is located in the device, between the bottom of the cartridge and the corresponding inner wall of the device. In one preferred embodiment the cartridge includes means for causing a spring action for pushing the stack of test strips or at least one strip in another arrangement towards the top side of the cartridge and the strip connectors face the side towards which the strip is pushed. This further assists in establishing a good contact between the test strip and electronics of the testing device.

A short description of the cartridge of PCT/FI/2010/050465 adapted to the invention is included below.

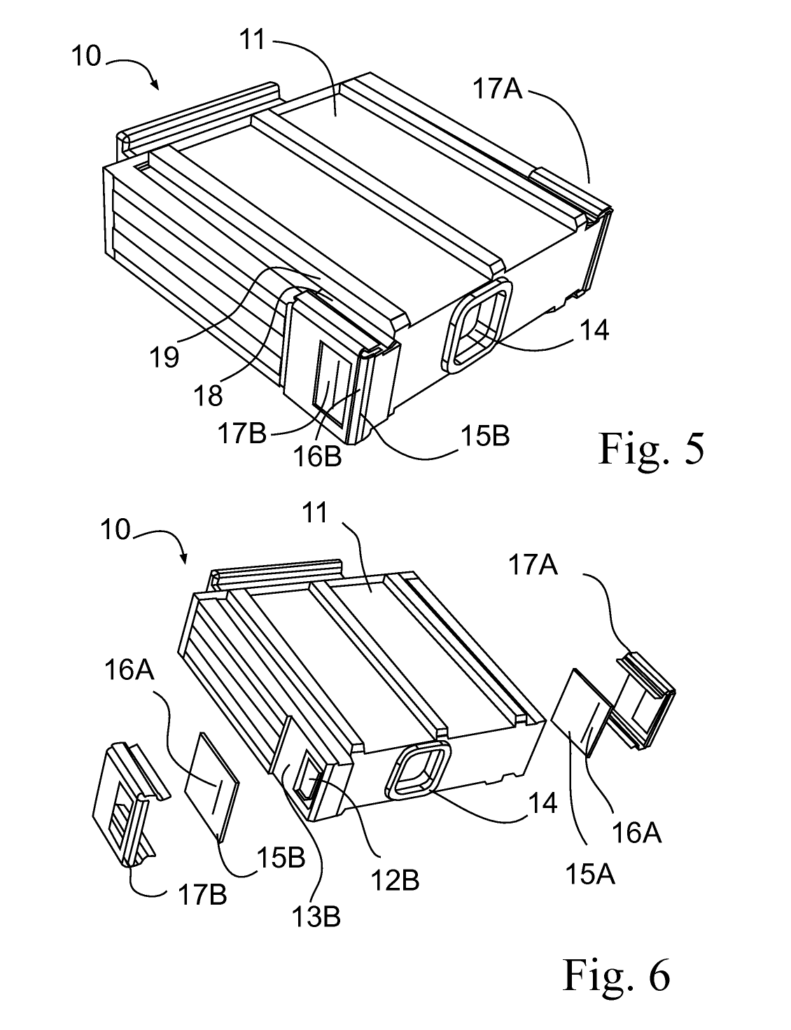

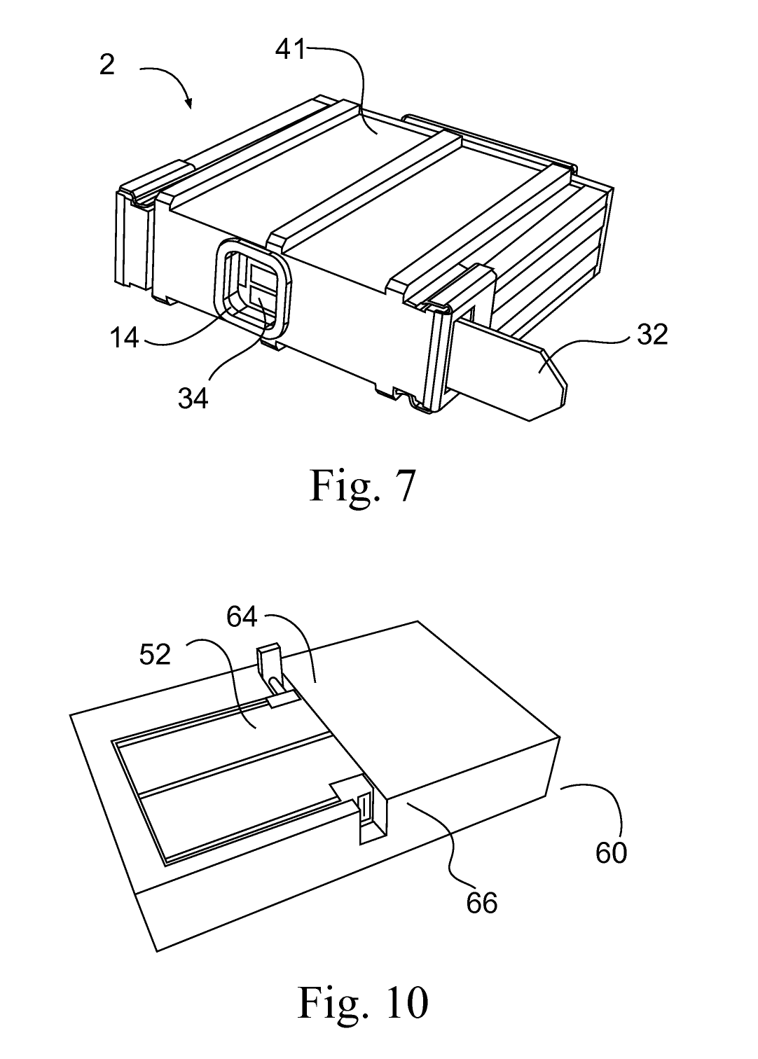

With reference to FIGS. 5-7, the cartridge 2 according to one embodiment comprises a generally rectangular body 41. On one face of the body 41, there is provided an opening 42B (“second opening”) for a strip pushing member (not shown) of a monitor device, and on an opposing face another opening (“first opening”, not shown in FIGS. 5 and 6) for a test strip 32. The openings are covered by elastic sheet-like members 15A, 15B, which can be tightly fitted onto respective flat zones 13B (the other fitting zone not shown) in the vicinity of the openings 42B (the other opening not shown) of the body 41. The elastic members 15A, 15B are provided with passages 16A, 16B which are normally closed but through which the test strips 32 or the pushing member can be pushed. The passages 16A, 16B are aligned with the openings 42B of the body.

The elastic members 15A, 15B are secured to the body 11 by means of retaining means, which take the form of clips 17A, 17B. When clipped on to the body, the clips 17A, 17B press the fringe areas of the elastic members 15A, 15B against the fitting zones 13B of the body 41. The clips 17A, 17B are shaped to have resilient clamps, which extend from the front faces of the clips 17A, 17B onto the sides of the body 41. The clamps are shaped to have shoulders, which are directed against each other. On corresponding sides of the body 41, there are provided grooves 19, to which the shoulders of the clamps engage. The clamps are designed such that after insertion, the clips 17A, 17B press the resilient members 15A, 15B towards the body 41 for achieving efficient sealing. The clips 17A, 17B also contain an openings on the front face thereof, the openings being aligned with the openings 12B and passages 16A, 16B and allowing the test strips 32 to exit the cartridge 1 or the pushing means to enter the cartridge. The clips 17A. 17B may also contain an additional flange on a side thereof, the flange serving to prevent slippage of the resilient members 15A, 15B.

The exemplary cartridge shown in FIGS. 1-3 also contains an opening 14 on the bottom face thereof. The opening 14 aids in electronic reading of the test strips 32. Thus, the test strips 32 contain reading terminals 34, which align with the opening 14 when being pushed partly out of the cartridge into a measurement position. The opening 14 may be in normal condition, i.e. when the test strip 32 is not in the measurement potion, sealed. The reading terminals 34 are exposed, for example, by the movement of the pushing means for the test strips 32. Thus, the opening 14 is preferably sealed before the cartridge is inserted into the meter but may be opened manually or automatically at insertion or during the use of the device.

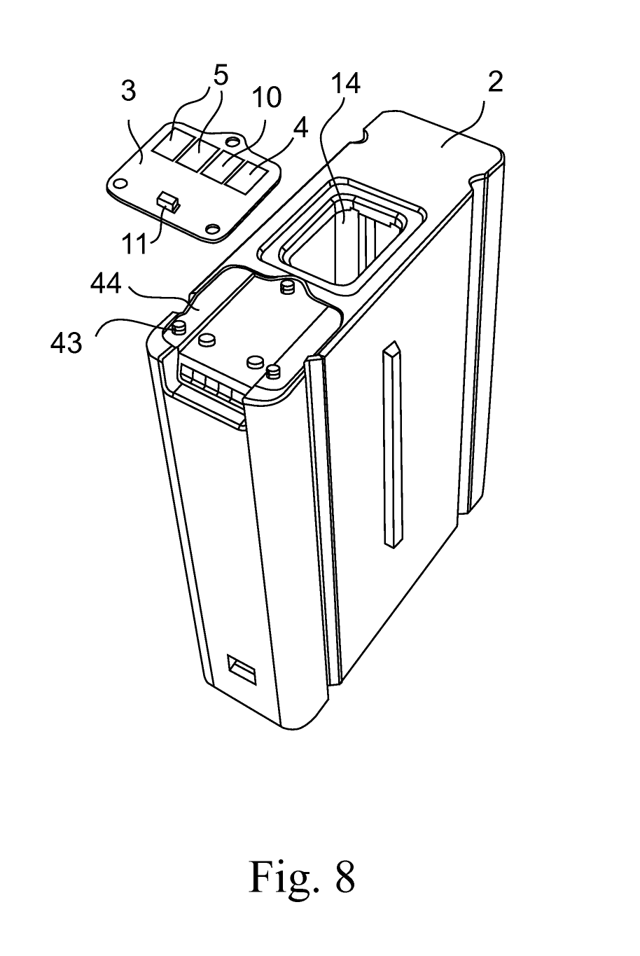

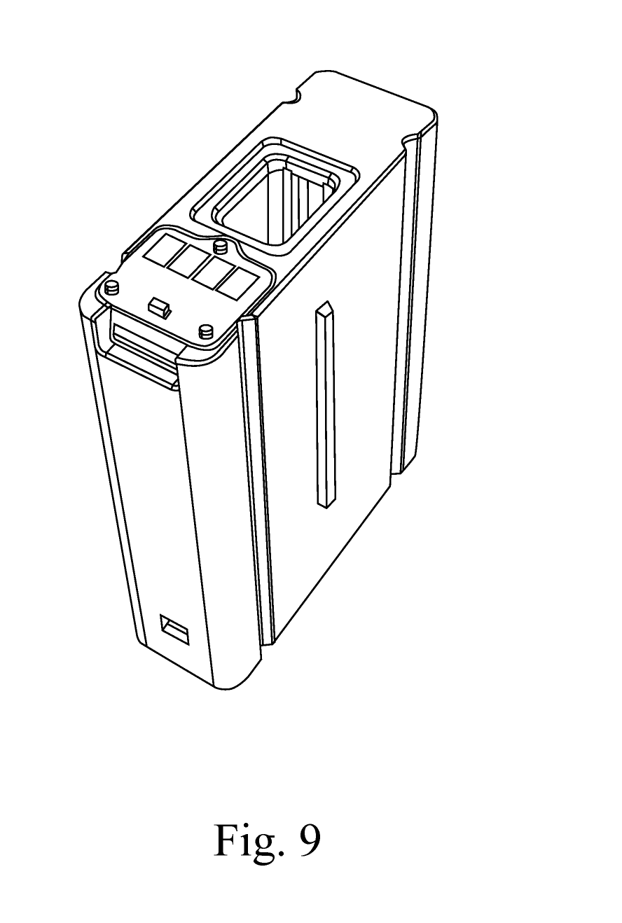

FIG. 8 shows the code plate 3 separate from the cartridge 2. The top part of the cartridge 2, which, for the purposes of this application, is the surface wherein the opening 14 for reading the test strips 32 is arranged, is provided with a recess 44 that has same dimensions as the outer perimeter of the code plate 3. Now the extension on the edge of the code plate 3 guides the plate 3 to a correct position and a possibility to misassemble is avoided. The cartridge has pegs 43 that are inserted into the holes 10 in the code plate 3 and sealed over the holes so that the code plate 3 and the cartridge are permanently joined together. This provides for easy coding of the cartridges simply by choosing a code plate that has a correct resistance corresponding to the desired code. The resistance 11 in the plate or the plate itself may have color coding in order to prevent attaching a wrong code plate.

Instead of resistance, another passive electric component or a memory chip or other memory device can be used. In each case choice of the coding element have various benefits and disadvantages. Using a resistor and the method for recognizing the resistance described in this application is presently consider especially beneficial.

The cartridge may also include means for the device to detect the insertion of the cartridge, means for memory of strip count, best-before dates and time since taking the cartridge to use. In a preferred embodiment, one or several of these means are integrated in the code plate. These functions may be accomplished by a re-writeable memory component in the cartridge, preferably in connection with the code plate, that is accessible by the meter.

The cartridge may be inserted in to a health monitoring device or other test strip dispensing device which contains a limited and substantially airtight space (not shown) to which the opening 14 communicates when the cartridge is inserted into the device. The boundary between the limited airtight space and the cartridge may comprise a gasket. Thus, even if the opening is in its open state, no ambient air can enter the cartridge. However, electrical contacting to the strips can take place through the limited airtight space and the opening. According to one embodiment, the electronic reading of test strips is carried out using conductors placed at the wall of the cartridge, thus forming an electronic pathway through the wall from the body fluid meter electronics to contact pads of the strip at measurement position. If the cartridge contains such conductors integral with the cartridge body, there are typically contact terminals provided on outer surface thereof. When inserted into the health-monitoring device or other test strip dispensing device, the contact terminals come into contact with reading means of the device.

FIG. 10 show a body fluid measuring apparatus equipped with a test strip cartridge. The apparatus comprises an apparatus body 60 having a space reserved for a strip cartridge 62. The apparatus comprises an actuator (plunger) 64 designed to fit from the second opening (not shown) of the cartridge 62 for engaging with a test strip and pushing the test strip out of the first opening 66. The apparatus may be provided with a second body part, i.e., a cover 61, for protecting the cartridge 62 and the actuator 64. On the surface of the body 60, there is provided interface means 68 for using the actuator 64. The actuator 64 may be spring-loaded.

According to one embodiment, means are provided at the vicinity of the second opening of the cartridge for transferring the movement of the external actuator to the test strips. That is, the actuator does not necessarily come into direct contact with the measurement strip to be pushed to measurement position but the movement may be transmitted by interface means.

The strip ejection actuator, which—when not in use—typically remains entirely outside the cassette. This arrangement enables the cartridge to be fully sealed when not in operation without complex integrated mechanism in the cassette. According to a preferred embodiment, the second opening is provided with identical sealing means, or at least operating with the same principle, than the exit opening of the strips.

Since it is of utmost importance that the electrical contacts between the connectors 4, 5, 34, in the code plate 2 and the test strip 32 with the connectors of the measuring apparatus do not fail, the cartridge is arranged to be pushed tightly against the top surface of the space in the apparatus body 60. The top of the cartridge 1 is maintained in tight contact with the measuring device by means such as a spring or tight mechanical fit.

It can also be contemplated that the connectors in the apparatus or even in the cartridge are equipped with resilient means such as springs or flexible pads for applying positive pressure between the connectors.

1. A cartridge for analytical test strips, comprising:
a body having a housing for a plurality of test strips,
a means for data transfer from the cartridge, and
resistor connected between the exactly two electrical connectors for identifying a calibration code for the analytical test strips included in the cartridge.

2. (canceled)

3. (canceled)

4. (canceled)

5. (canceled)

6. The cartridge according to claim 1, wherein the cartridge has test strips arranged in a stack format.

7. The cartridge according to claim 1, wherein the cartridge has means to facilitate electrical contact with the test strip while the strip is partially in the cartridge.

8. The cartridge according to claim 7, wherein the means for identifying the calibration code and the means for electrical contact to the test strip are arranged on the same, top surface of the cartridge.

9. The cartridge according to claim 1, wherein the cartridge has at least one opening on a side wall for dispensing a test strip.

10. The cartridge according to claim 1, wherein the cartridge has another opening on the opposite side to facilitate pushing out of a test strip.

11. The cartridge according to claim 1, wherein the movement of the strip is arranged to cause the strip to make contact with the electronics of an analyzing device.

12. The cartridge according to claim 1, wherein the fitting between the cartridge and an analyzing device is designed so that when the cartridge is placed in the device, the fitting facilitates a pressure to provide good contact with the strip and coding connectors to the device.

13. The cartridge according to claim 1, wherein the cartridge has means for pushing the strips included in the cartridge so that the at least one strip included in the cartridge is pushed towards the top side of the cartridge and the top of the cartridge has means to limit the upward movement.

14. The cartridge according to claim 1, wherein the cartridge has an opening on which the test strip can be placed when it is at least partially pushed out of the cartridge so that the contact pads of the strip are placed in contact with the corresponding connectors of the analyzing device through the opening.

15. (canceled)

16. The cartridge according to the claim 1, wherein the electrical connectors and the resistor are arranged in a code plate that is a separate body to the cartridge and the code plate is attached to the cartridge by a permanent manner.

17. The cartridge according to claim 16, wherein the code plate is a printed wiring board (PWB).

18. The cartridge according to claim 1, further comprising means provided in the cartridge for enabling the device to detect the insertion of the cartridge through the connections of the code plate.

19. The cartridge according to claim 1, wherein the cartridge has a memory means in connection with the code plate, to store data related to the test strips and their use and means to communicate these with the device.

20. The cartridge according to claim 19, wherein the memory means is re-writeable and the device has means for updating the contents of the memory.

21. The cartridge according to claim 19, wherein the data related to the test strips is a best-before date.

22. The cartridge according to claim 19, wherein the data related to the test strips is strip count.

23. The cartridge according to claim 19, wherein the data related to the test strips is the time when the cartridge was first taken into use.

24. The cartridge according to claim 19, wherein the data related to the test strips is the amount of time since taking the cartridge into use.

25. A method for including calibrating code to a multiple cartridges for analytic test strips, each of the cartridges having a body having a housing for a plurality of test strips, said method comprising the steps of:
providing two electrical connectors and a resistor connected between the electrical connectors on the housing of the cartridge for identifying a calibration code for the lot of strips included in the cartridge, and
providing the multiple cartridges with resistors having resistance values that are multiplies of each other.

26. A method for recognizing a code for a cartridge for analytical test strips, said method comprising the steps of:
connecting voltage from an output over a first resistance (Rcode or Rref)
determining the first time (time (code)/time(ref)) needed for the voltage to rise to a set level at input,
determining a second time (time(ref)/time(code)) needed for the voltage to rise over a second resistance,
determining, on basis of resistance that is known, and the relation between first and second time, the unknown resistance, the unknown resistance being placed in the cartridge, and
determining the calibration code on basis of the measured value of the unknown resistance.

27. The method according to claim 26, wherein the resistance values of the unknown resistances (Rcode) are set to be multiplies of the known resistance (Rref).

28. The method according to claim 26, further comprising the step of connecting the first resistance (Rref) and the second resistance (Rcode) between one input (Input) of a microcontroller (uC) and separate outputs (Output(code), Output(ref)) through one capacitator.

29. An arrangement for recognizing a code for a cartridge for analytical test strips, comprising a device (uC) for calculating time and providing voltage between at least two outputs (Output(code), Output(ref)) and one input (Input),
a resistance (Rref) and a capacitance (C) connected to a first output electrical connector (output(ref)) and an input electrical connector (Input),
at least one electrical connector connected to second output (Output(code)) and one electrical connector connected to input (Input) and capacitance (C), and
at least two electrical connectors arranged on the cartridge and joined by a resistance, the at least two electrical connectors being capable of connecting the resistance (Rcode) between the second output (Output(code) and the input (Input) and to the capacitance (C).

30. An arrangement according to claim 29, wherein the device is a microcontroller and the outputs and input are three pins therein.

31. The arrangement according to claim 29, wherein the electrical connections and the resistance of the cartridge are set on a separate code plate attached to the cartridge.

32. The arrangement according to claim 31, wherein the code plate is formed of PWB.
